(19) United States
(12) Patent Application Publication
(10) Pub. No.: US 2015/0362384 A1
(43) Pub. Date: Dec. 17, 2015

(54) METHOD FOR DETERMINING SENSING VALUE AND ELECTRONIC DEVICE PERFORMING THE SAME

(71) Applicant: Samsung Electronics Co., Ltd., Gyeonggi-do (KR)

(72) Inventor: In Ho PARK, Gyeonggi-do (KR)

(73) Assignee: Samsung Electronics Co., Ltd.

(21) Appl. No.: 14/737,108

(22) Filed: Jun. 11, 2015

(30) Foreign Application Priority Data
Jun. 11, 2014 (KR) 10-2014-0070935

(51) Int. Cl. G01K 15/00; G01N 33/00

(52) U.S. Cl. CPC G01K 15/005; G01N 33/0006

(57) ABSTRACT

Methods and apparatuses are provided for determining a sensing value. An electronic device receives, from an external device, a reference sensing value related to a sensor of the electronic device. An initial sensing value is determined by measuring a parameter with the sensor. It is determined whether the initial sensing value is within an error range of the reference sensing value. The initial sensing value is compensated with the reference sensing value, if the initial sensing value is not within the error range. The sensor determines a sensing value based on the compensated sensing value. The sensing value is repeatedly determined until the sensing value is within the error range or until a set time expires.

### PRIORITY

This application claims priority under 35 U.S.C. §119(a) to a Korean Patent Application filed on Jun. 11, 2014 in the Korean Intellectual Property Office and assigned serial number 10-2014-0070935, the content of which is incorporated herein by reference.

### BACKGROUND OF THE INVENTION

1. Field of the Invention

The present invention relates generally to an electronic device, and more particularly, to a method for determining a sensing value and an electronic device for performing the same.

2. Description of the Related Art

Portable mobile devices, such as smartphones, are equipped with various sensors that enable users to recognize environmental conditions. For example, various portable mobile devices are equipped with sensors for measuring temperature or humidity.

Sensors that are installed within a portable mobile device may be affected by the temperature in the device, the temperature of a human body contacting the device, and/or environmental conditions such as, for example, operation of a heater or an air conditioner, resulting in inaccurate sensing values. Furthermore, a long measurement time is required to produce meaningful measurement values.

### SUMMARY OF THE INVENTION

The present invention has been made to address at least the above problems and/or disadvantages and to provide at least the advantages described below. Accordingly, an aspect of the present invention provides a method for determining a sensing value and an electronic device for performing the same.

Another aspect of the present invention provides a computer-readable recording medium for recording a program for performing the method in a computer.

In accordance with an aspect of the present invention, a method is for determining a sensing value. An electronic device receives, from an external device, a reference sensing value related to a sensor of the electronic device. An initial sensing value is determined by measuring a parameter with the sensor. It is determined whether the initial sensing value is within an error range of the reference sensing value. The initial sensing value is compensated with the reference sensing value, if the initial sensing value is not within the error range. The sensor determines a sensing value based on the compensated sensing value. The sensing value is repeatedly determined until the sensing value is within the error range or until a set time expires.

In accordance with another aspect of the present invention, a computer-readable recording medium for recording a program is provided for performing a method of determining a sensing value in a computer. The method comprises the steps of: receiving, at an electronic device, from an external device, a reference sensing value related to a sensor of the electronic device; determining an initial sensing value by measuring a parameter with the sensor; determining whether the initial sensing value is within an error range of the reference sensing value; compensating the initial sensing value with the reference sensing value, if the initial sensing value is not within the error range; determining, by the sensor, a sensing value based on the compensated sensing value; and repeatedly determining the sensing value until the sensing value is within the error range or until a set time expires.

In accordance with another aspect of the present invention, an electronic device is provided and includes a sensor configured to determine a sensing value by measuring a parameter. The electronic device also includes a communication interface unit configured to receive a reference sensing value related to the sensor from an external device. The electronic device further includes a control unit configured to determine whether an initial sensing value determined by the sensor is within an error range of the reference sensing value. The control unit is also configured to compensate the initial sensing value with the reference sensing value, if the initial sensing value is not within the error range. The control unit is further configured to control the sensor to repeatedly determine the sensing value based on the compensated sensing value until the sensing value is within the error range or until a set time expires.

In accordance with another aspect of the present invention, a sensor is provided for determining a sensing value. The sensor includes a measurement unit configured to measure a parameter. The sensor also includes a measurement processing unit configured to determine an initial sensing value of the measured parameter. The sensor additionally includes a calibration unit configured to compensate the initial sensing value with a reference sensing value, if the initial sensing value is not within an error range of the reference sensing value. The measurement processing unit repeatedly determines a sensing value based on the compensated sensing value until the sensing value is within the error range or until a set time expires.

### BRIEF DESCRIPTION OF THE DRAWINGS

The above and other aspects, features, and advantages of the present invention will be more apparent from the following detailed description when taken in conjunction with the accompanying drawings, in which:

FIG. 1 is a block diagram illustrating an electronic device, according to an embodiment of the present invention;

FIG. 2 is a block diagram illustrating a mobile terminal, according to an embodiment of the present invention;

FIG. 3 is a block diagram illustrating a sensor, according to an embodiment of the present invention;

FIG. 4 is a flowchart illustrating a method for determining a sensing value, according to an embodiment of the present invention; and

FIG. 5 is a flowchart illustrating a method for determining a sensing value, according to an embodiment of the present invention.

### DETAILED DESCRIPTION OF EMBODIMENTS OF THE PRESENT INVENTION

Embodiments of the present invention are described with reference to the accompanying drawings. The same or similar components may be designated by the same or similar reference numerals although they are illustrated in different drawings. Detailed descriptions of constructions or processes known in the art may be omitted to avoid obscuring the subject matter of the present invention.

The terms “include,” “comprise,” “including,” or “comprising”, as used herein, indicate disclosed functions, operations, or existence of elements, but does not exclude other functions, operations, or elements. It should be further understood that the terms “include”, “including”, “comprise”, “comprising”, “have”, or “having”, as used herein, specify the presence of stated features, numbers, operations, elements, components, or combinations thereof, but do not preclude the presence or addition of one or more other features, numbers, operations, elements, components, or combinations thereof.

The meanings of the terms “or” or “at least one of A and B”, as used herein, include any and all combinations of words listed together with the term. For example, the wording “A or B” or “at least one of A and B” may indicate A, B, or both A and B.

Terms such as “first”, “second”, and the like, as used herein, may refer to various elements of various embodiments of the present disclosure, but do not limit the elements. For example, such terms do not limit the order and/or priority of the elements. Furthermore, such terms may be used to distinguish one element from another element. For example, a first user device and a second user device indicate different user devices. Additionally, without departing the scope of the present invention, a first element may be referred to as a second element, and similarly, a second element may be referred to as a first element.

It will be understood that when an element is referred to as being “connected” or “coupled” to another element, it can be directly connected or coupled to the other element or intervening elements may be present. In contrast, when an element is referred to as being “directly connected” or “directly coupled” to another element, it should be understood that there are no intervening elements therebetween.

The terminology used herein is not for delimiting the present disclosure, but instead for describing specific various embodiments of the present disclosure. The terms of a singular form may include plural forms unless otherwise specified.

The terms used herein, including technical or scientific terms, have the same meanings as understood by those skilled in the art unless otherwise defined herein. The commonly used terms, such as those defined in a dictionary, should be interpreted in the same context as in the related art and should not be interpreted in an idealized or overly formal detect unless otherwise defined explicitly.

Electronic devices, according to various embodiments of the present invention, may have a communication function. For example, the electronic devices may be embodied as at least one of smartphones, tablet personal computers (PCs), mobile phones, video telephones, electronic book readers, desktop PCs, laptop PCs, netbook computers, personal digital assistants (PDAs), portable multimedia players (PMPs), Motion Picture Experts Group (MPEG-1 or MPEG-2) Audio Layer 3 (MP3) players, mobile medical devices, cameras, and wearable devices (e.g., head-mounted-devices (HMDs) such as electronic glasses, an electronic apparel, electronic bracelets, electronic necklaces, electronic appcessories, electronic tattoos, or smart watches).

According to various embodiments of the present invention, the electronic devices may be embodied as smart home appliances having a communication function. The smart home appliances may include at least one of, for example, televisions (TVs), digital versatile disc (DVD) players, audio players, refrigerators, air conditioners, cleaners, ovens, microwave ovens, washing machines, air cleaners, set-top boxes, TV boxes, game consoles, electronic dictionaries, electronic keys, camcorders, electronic picture frames, and the like.

According to various embodiments of the present invention, the electronic devices may be embodied as at least one of medical devices (e.g., a magnetic resonance angiography (MRA), a magnetic resonance imaging (MRI), a computed tomography (CT), scanners, and ultrasonic devices), navigation devices, global positioning system (GPS) receivers, event data recorders (EDRs), flight data recorders (FDRs), vehicle infotainment devices, electronic equipment for ships (e.g., navigation systems and gyrocompasses), avionics, security devices, head units for vehicles, industrial or home robots, automatic teller's machines (ATMs), and points of sales (POSs).

According to various embodiments of the present invention, the electronic devices may be embodied as at least one of parts of furniture or buildings/structures, electronic boards, electronic signature receiving devices, projectors, and measuring instruments (e.g., water meters, electricity meters, gas meters, or wave meters) having a communication function. The electronic devices according to various embodiments of the present invention may be one or more combinations of the above-described devices. Furthermore, the electronic devices according to various embodiments of the present invention may be flexible devices. It would be obvious to those skilled in the art that the electronic devices, according to various embodiments of the present invention, are not limited to the above-described devices.

The term “user” used herein may refer to a person who uses an electronic device or may refer to a device (e.g., an artificial electronic device) that uses the electronic device.

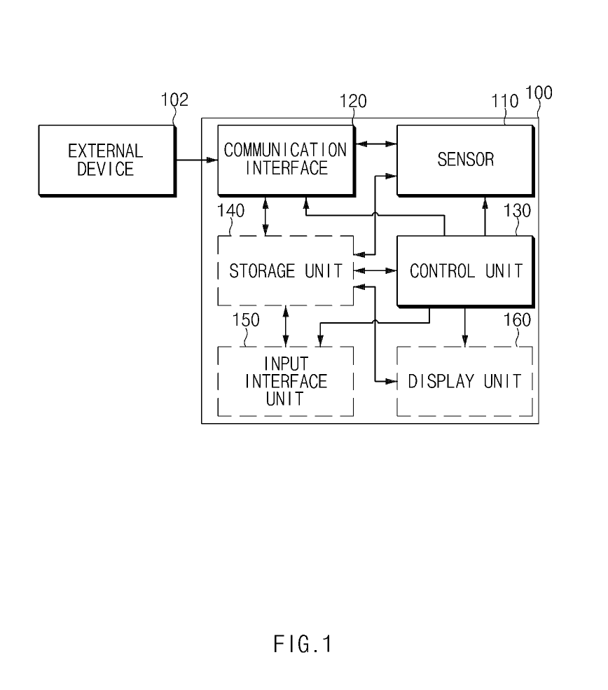

FIG. 1 is a block diagram illustrating an electronic device, according to an embodiment of the present invention. Referring to FIG. 1, an electronic device 100 includes a sensor 110, a communication interface unit 120, and a control unit 130. In an embodiment of the present invention, the electronic device 100 further includes at least one of a storage unit 140, an input interface unit 150, and a display unit 160.

In order not to unnecessarily obscure the features of this embodiment of the present invention, only relevant elements are described in detail below. Those skilled in the art would understand that general-purpose elements may be further included in addition to the elements illustrated in FIG. 1.

The electronic device 100 may accurately and quickly determine a sensing value of the sensor 110 using a reference sensing value received from an external device 102. The term “sensing value”, as used herein, may be construed as a value of a parameter measureable by the sensor 110. For example, a value of a temperature in a current location measurable by a temperature sensor may correspond to a sensing value.

A sensing value determined by the sensor 110 of the electronic device 100 may rapidly change due to an increase in an internal temperature, a temperature of a human body contacting the electronic device 100, or environmental conditions, such as, for example, operation of a heater or an air conditioner. In order to reduce the influence of the above-described factors, the electronic device 100 may repeatedly determine a sensing value until a stable (or meaningful) sensing value is obtained. In order to confirm a sensing value within a short time, the electronic device 100 may repeatedly determine a sensing value for a set time.

The electronic device 100 may notify a user of a sensing value obtained after the set time has elapsed, as a final sensing value. Through repeated determination, the electronic device 100 may obtain the final sensing value that is an accurate approximation of an actual value. The term “actual value”, as used herein, may represent a sensing value determined without being affected by the internal temperature increase, the human body temperature, the environmental conditions, or the like.

For convenience, a sensing value obtained by the electronic device 100 after the set time has elapsed is hereinafter referred to as a final sensing value. The final sensing value may be differentiated from a sensing value obtained while the sensing value is repeatedly determined. The electronic device 100 may output the final sensing value to the user through the display unit 160.

The electronic device 100 may obtain the final sensing value by repeatedly determining a sensing value for the set time. The electronic device 100 may perform the determination process whenever the user requests a sensing value, and the electronic device 100 may output the final sensing value obtained through the determination process.

As a difference between an actual value and a sensing value initially determined through the determination process becomes smaller, the electronic device 100 may more quickly obtain the final sensing value that is more accurate with respect to the actual value. For convenience, the sensing value initially determined through the determination process is hereinafter referred to as an initial sensing value. The initial sensing value may be differentiated from a sensing value obtained while the sensing value is repeatedly determined.

If the electronic device 100 receives a sensing value and compensates the initial sensing value with the sensing value, the electronic device 100 may more quickly obtain the final sensing value more accurate with respect to the actual value. The electronic device 100 may receive the reference sensing value and compensate the initial sensing value with the reference sensing value so as to obtain a more accurate final sensing value.

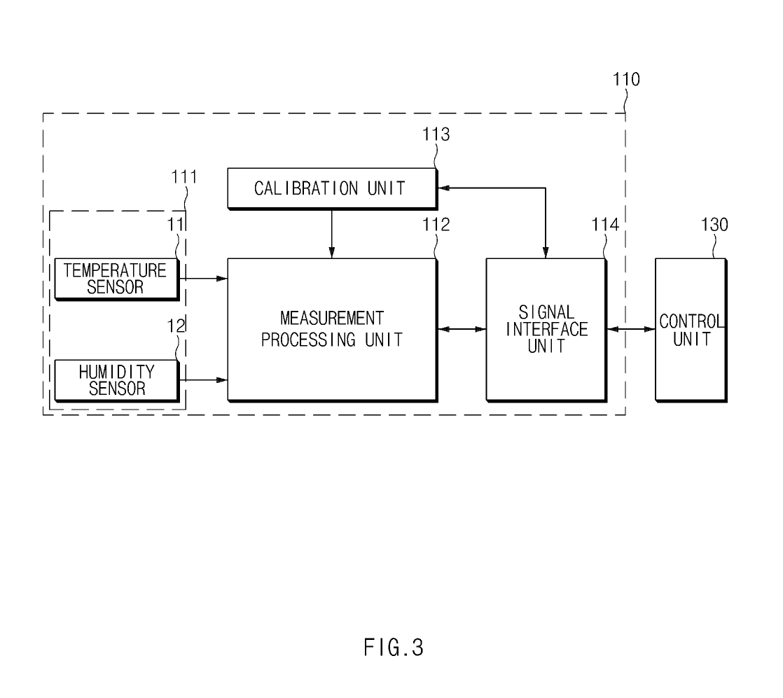

The sensor 110 may determine a sensing value. According to various embodiments of the present invention, if the sensor 110 is a temperature sensor, the sensing value may represent a temperature. Alternatively, if the sensor 110 is a humidity sensor, the sensor value may represent humidity. The sensor 110 a may be at least one of a temperature sensor, a humidity sensor, and a temperature-humidity sensor. However, the sensor 110 is not limited thereto, and may further include various sensors such as an illumination sensor, an ultraviolet sensor, a barometric pressure sensor, or the like. The sensor 110 is described in greater detail below with reference to FIG. 3.

The communication interface unit 120 may receive, from the external device 102, a reference sensing value related to the sensor 110. For example, if the sensor 110 is a temperature-humidity sensor, the communication interface unit 120 may receive a reference temperature value together with a reference humidity value from the external device 102.

The reference sensing value may vary with a location or space in which the electronic device 100 is positioned or a date or time requested by the electronic device 100. Therefore, the reference sensing value may be used to compensate an initial sensing value for a date, a time slot, an area (location), or a space in which a determination is to be performed in the electronic device 100.

According to an embodiment of the present invention, the communication interface unit 120 may transmit location information of the electronic device 100 to the external device 102. Accordingly, the communication interface unit 120 may receive, from the external device 102, the reference sensing value determined based on the location information of the electronic device 100.

According to an embodiment of the present invention, the communication interface unit 120 may transmit, to the external device 102, a final sensing value determined by the electronic device 100. The external device 102 may use the received final sensing value as the reference sensing value. The external device 102 may provide the final sensing value received from the electronic device 100 as the reference sensing value to another electronic device that is located in the same area or space as that of the electronic device 100 and that has requested the reference sensing value at the same date as or similar time slot to that of the electronic device 100.

According to an embodiment of the present disclosure, the electronic device 100 may directly transmit the final sensing value as the reference sensing value to another electronic device positioned within a specified distance from the electronic device 100.

The communication interface unit 120 may transmit/receive data via a wired/wireless network or wired serial communication. The network includes the Internet, a local area network (LAN), a wireless LAN, a wide area network (WAN), a personal area network (PAN), or the like, but is not limited thereto. It is understood by those skilled in the art that the network may be any other network for transmitting/receiving information.

According to an embodiment of the present invention, the communication interface unit 120 may transmit/receive data to/from the external device 102 based on a short-range communication technology. The short-range communication technology according to the present embodiment may include Bluetooth, radio frequency identification (RFID), infrared data association (IrDA), ultra wideband (UWB), ZigBee, Wi-Fi direct (WFD), near field communication (NFC), or the like.

The control unit 130 may control the sensor 110 so that the sensor 110 may determine a sensing value approximate to an actual value within a set time.

According to an embodiment of the present invention, the control unit 130 may determine whether the initial sensing value determined by the sensor 110 is within a specified error range from the reference sensing value. The control unit 130 may determine whether to compensate the initial sensing value with the reference sensing value, according to whether the initial sensing value determined by the sensor 110 is within the error range. The error range may be a value preset in the electronic device 100 or may be set by the user in various ways.

If the initial sensing value is within the error range from the reference sensing value, the control unit 130 may not compensate the initial sensing value with the reference sensing value. Since the initial sensing value is within the error range, the sensor 110 may determine the sensing value, which is an accurate approximate of the actual value, within a set time. Therefore, the control unit 130 may determine that the reference sensing value is not required to be used. Accordingly, the control unit 130 may control the sensor 110 so that the final sensing value is determined based on the initial sensing value after a set time has elapsed. The set time represents a time taken to obtain the final sensing value by repeatedly determining a sensing value through the determining process. The set time may be a value preset in the electronic device 100 or may be set by the user in various ways.

According to an embodiment of the present invention, if the initial sensing value is outside the error range from the reference sensing value, the control unit 130 may compensate the initial sensing value with the reference sensing value. By compensating the initial sensing value with the reference sensing value, the control unit 130 may reduce a range of a determined sensing value, which varies physically. Therefore, a time for the sensor 110 to arrive at a sensing value, which is an accurate approximation of an actual value, may be reduced.

The control unit 130 may control, based on a compensated sensing value, the sensor 110 so that the sensor 110 repeatedly determines a sensing value until a determined sensing value is within the error range or a set time expires. According to an embodiment of the present invention, the control unit 130 may repeat determination of a sensing value at an interval of a specified time. According to an embodiment of the present invention, the interval of the specified time may be a value preset in the electronic device 100 or may be set by the user in various ways.

According to an embodiment of the present invention, if a determined sensing value moves within the error range, the control unit 130 may determine that the sensing value determined by the sensor 110 is an accurate approximate of the actual value, and may terminate a process of repeatedly determining the sensing value. After the set time has elapsed, the control unit 130 may control the sensor 110 so that the sensor 110 determines the final sensing value based on the sensing value most recently determined.

According to an embodiment of the present invention, if the set time expires, the control unit 130 may terminate the determination process of repeatedly determining the sensing value. The control unit 130 may determine that the actual value is different from the reference sensing value to a certain degree, and may determine the most recently determined sensing value as the final sensing value.

According to an embodiment of the present invention, if a difference between a determined sensing value and the reference sensing value becomes greater than that between a previously determined sensing value and the reference sensing value, the control unit 130 may determine that initial compensation of the reference sensing value is not effective. Therefore, the control unit 130 may discard the reference sensing value by changing the compensated sensing value into the initial sensing value not compensated. The control unit 130 may control the sensor 110 so that the sensor 110 determines the final sensing value after the set time has elapsed based on the initial sensing value not compensated with the reference sensing value.

The control unit 130 may include at least one processor.

The storage unit 140, which is a typical storage medium may store data or a program required for controlling sensing value determination of the sensor 110. Furthermore, the storage unit 140 may further include data or a program required for the control unit 130 to determine whether the initial sensing value is within the error range of the reference sensing value, whether to compensate the initial sensing value with the reference sensing value, or whether a determined sensing value is within the error range.

According to an embodiment of the present invention, the storage unit 140 may include a program routine or an instruction set required for the control unit 130 to control the sensor 110, the communication interface unit 120, the input interface unit 150, or the display unit 160.

According to an embodiment of the present invention, the storage unit 140 may store data or a program for operating the electronic device 100. The storage unit 140 may be implemented with a hard disk drive (HDD), a read only memory (ROM), a random access memory (RAM), a flash memory, a memory card, a NAND memory, a solid state drive (SSD), or the like.

The input interface unit 150 may receive input information input by the user.

According to an embodiment of the present invention, the input interface unit 150 may receive input from the user regarding whether to use the reference sensing value. For example, if the user determines that use of the reference sensing value is meaningless, the user may select an option not to use the reference sensing value. Therefore, if the input interface unit 150 receives this selection, the control unit 130 may control the sensor 110 so that the reference sensing value is not used.

According to an embodiment of the present invention, the input interface unit 150 may receive, from the user, the error range for the sensor 110. For example, the user may directly input, through the input interface unit 150, a desired error range for a sensing value to be determined. The control unit 130 may control the sensor 110 so that the sensor 110 determines a sensing value using the input error range.

According to an embodiment of the present invention, the input interface unit 150 may receive, from the user, a time taken to perform the determination process or a time interval between determinations of a sensing value. For example, if the user desires to quickly check a sensing value, the user may input, through the input interface unit 150, a time or a time interval shorter than a currently set time or time interval. The control unit 130 may control the sensor 110 so that the sensor 110 determines a sensing value using the input time or time interval. The input interface unit 150 may include an input device such as a keypad, a dome switch, a touchpad (resistive/capacitive type), a jog wheel, a jog switch, an H/W button, or the like, and a software module for driving the input device.

The display unit 160 may output, to the user, information processed in the electronic device 100 in various formats such as a text, an image, a video, or the like.

According to an embodiment of the present invention, the display unit 160 may convert the final sensing value into another format to output the final sensing value. The display unit 160 may show, to the user, the final sensing value in the format of multimedia data or text data through a display device. Alternatively, the electronic device 100 may convert the final sensing value into voice data and may output the voice data to the user through a speaker.

The display unit 160 may include an output device such as a touch screen, a liquid crystal display (LCD), a thin-film transistor LCD, an organic light-emitting diode (OLED), a flexible display, a 3D display, or the like, and a software module for driving the output device.

According to an embodiment of the present invention, the external device 102 may store the reference sensing value, and may communicate with the electronic device 100 to transmit the reference sensing value thereto. According to an embodiment of the present invention, the external device 102 may receive a request for the reference sensing value from the electronic device 100.

According to an embodiment of the present invention, the external device 102 may be a server. The server may collect the reference sensing values for each area, date, time slot, and space, and may provide the collected reference sensing values to the electronic device 100.

The external device 102 may be a server, but is not limited thereto. The external device 102 may include any electronic device capable of communicating with the electronic device 100 such as a mobile phone, a smartphone, a notebook computer, a terminal for a digital broadcast, a digital camera, a portable game terminal, a PDA, a PMP, a navigator, a tablet PC, and the like.

According to an embodiment of the present invention, the external device 102 may be a mobile terminal device similar to the electronic device 100. The external device 102 may determine the final sensing value in the same manner as that of the electronic device 100, and may transmit the final sensing value as the reference sensing value to an electronic device positioned within a specified distance from the external device 102.

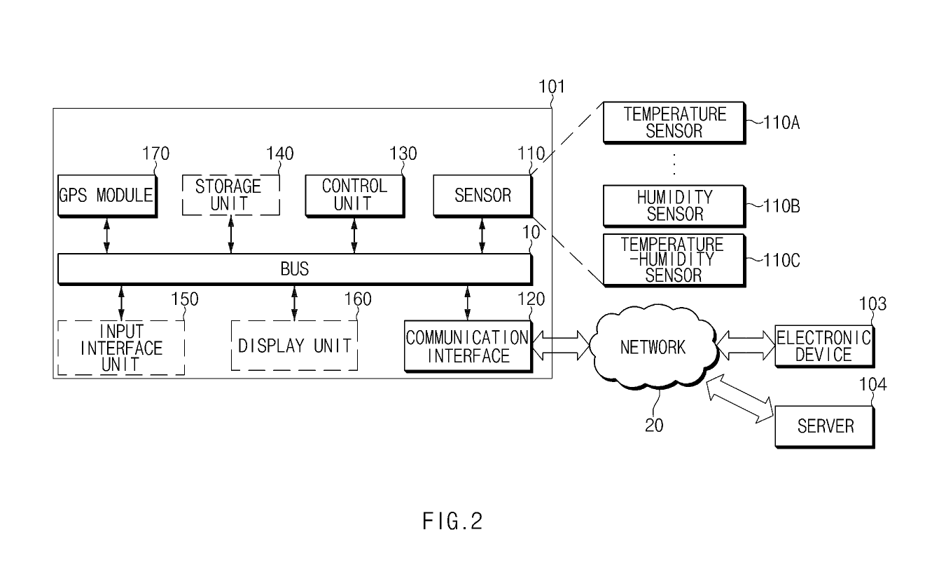

FIG. 2 is a block diagram illustrating a mobile terminal, according to an embodiment of the present invention. A mobile terminal 101 of FIG. 2 may be an example of the electronic device 100. Referring to FIG. 2, the mobile terminal 101 includes a bus 10, the sensor 110, the communication interface unit 120, the control unit 130, the storage unit 140, the input interface unit 150, the display unit 160, and a GPS module 170.

In order not to unnecessarily obscure the features of this embodiment of the present invention, only relevant elements are described in detail below. Those skilled in the art understand that general-purpose elements may be further included in addition to the elements illustrated in FIG. 2.

The mobile terminal 101 may accurately and quickly determine a sensing value of the sensor 110 using the reference sensing value received from an electronic device 103 or a server 104.

The sensor 110 may determine a sensing value. The sensor 110 according to the present embodiment includes at least one of a temperature sensor 110A, a humidity sensor 110B, and a temperature-humidity sensor 110C. However, the sensor 110 is not limited thereto, and may further include any sensor for measuring a state change.

The communication interface unit 120 may receive, from the electronic device 103 or the server 104, the reference sensing value of the sensor 110. The reference sensing value may vary with a location or space in which the mobile terminal 101 is positioned, or a date or time requested by the mobile terminal 101.

According to an embodiment of the present invention, the communication interface unit 120 may receive the reference sensing value from the electronic device 103 associated with the mobile terminal 101 based on various communication technologies, such as, for example, Bluetooth, RFID, IrDA, UWB, ZigBee, WFD, NFC, or the like. According to an embodiment of the present invention, the communication interface unit 120 may receive the reference sensing value from the server 104 via a wired/wireless network or wired serial communication.

The GPS module 170 may obtain location information of the mobile terminal 101 when the reference sensing value is requested. The location information may be obtained in the form of latitude, longitude, altitude, speed, azimuth, or the like.

The server 104 may collect the reference sensing values for each area, date, time slot, and space, and may provide the reference sensing values to the mobile terminal 101. According to an embodiment of the present invention, the server 104 may be a cloud server.

FIG. 3 is a block diagram illustrating a sensor, according to an embodiment of the present invention. Referring to FIG. 3, the sensor 110 includes a measurement unit 111, a measurement processing unit 112, a calibration unit 113, and a signal interface unit 114.

The measurement unit 111 may obtain sensing data (e.g., a sensing value). According to whether the sensor 110 is a temperature sensor or a humidity sensor, the measurement unit 111 includes a temperature sensor 11 or a humidity sensor 12. When the sensor 110 is a temperature-humidity sensor, the measurement unit 111 includes both the temperature sensor 11 and the humidity sensor 12.

The measurement processing unit 112 may perform signal processing on the sensing data obtained by the measurement unit 111. For example, the measurement processing unit 112 may include an amplifier, an analog-to-digital (A/D) converter, or the like. Therefore, the measurement processing unit 112 may amplify the sensing data detected by the temperature sensor 11 or the humidity sensor 12. The measurement processing unit 112 may convert the sensing data obtained as analog data by the temperature sensor 11 or the humidity sensor 12 into digital data.

According to an embodiment of the present invention, the measurement processing unit 112 may compensate the initial sensing value of the sensor 110 according to a control signal of the control unit 130.

The calibration unit 113 may compensate the initial sensing value through calibration.

The signal interface unit 114 may transfer a signal between the control unit 130 and the measurement processing unit 112, or may transfer a signal between the control unit 130 and the calibration unit 113. For example, when the control unit 130 compensates the initial sensing value of the sensor 110, the signal interface unit 114 may compensate the measurement processing unit 112 by software from the control unit 130. Alternatively, the signal interface unit 114 may receive calibration information from the control unit 130 to compensate the initial sensing value of the sensor 110 through the calibration unit 113.

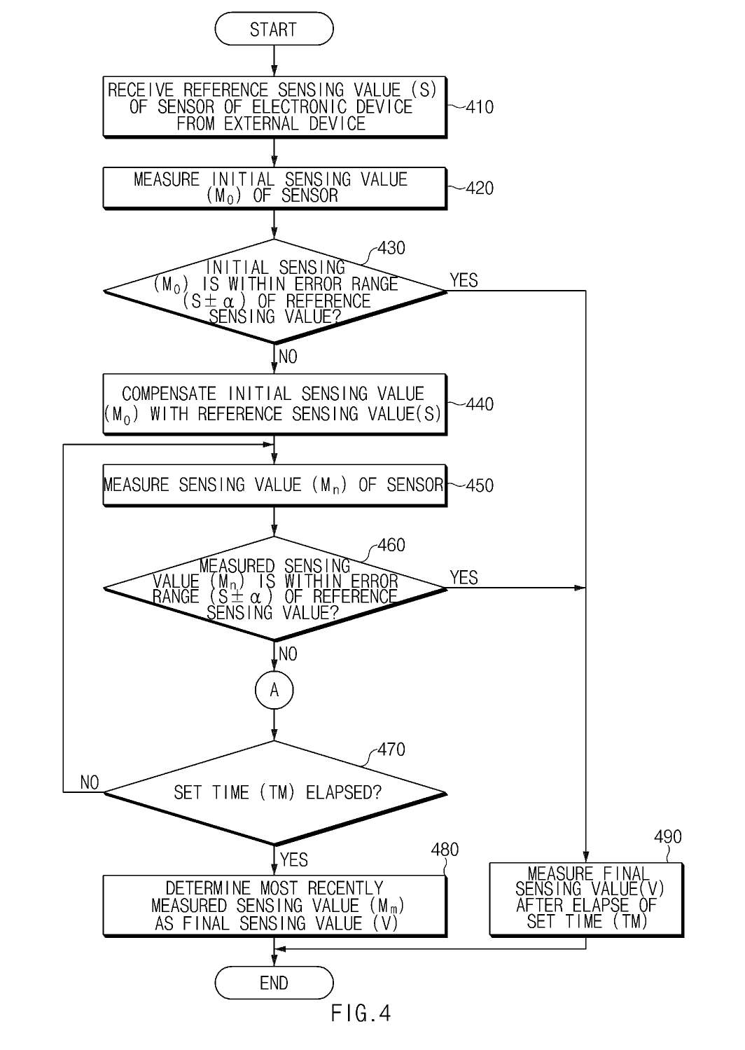

FIG. 4 is a flowchart illustrating a method for determining a sensing value, according to an embodiment of the present invention. The flowchart of FIG. 4 may include operations performed in the electronic device 100 or the mobile terminal 101 illustrated in FIGS. 1 to 3. Therefore, the above descriptions of the electronic device 100 or the mobile terminal 101 illustrated in FIGS. 1 to 3 may be applied to the flowchart illustrated in FIG. 4.

In step 410, the sensor 110 receives, from the external device 102, a reference sensing value S of the sensor 110 of the electronic device 100. The reference sensing value S may vary with a location or space in which the electronic device 100 is positioned, or with a date or time requested by the electronic device 100.

In step 420, the sensor 110 determines an initial sensing value M₀.

In step 430, the control unit 130 determines whether the initial sensing value M₀ is within an error range S±α of the reference sensing value S. The control unit 130 determines whether to compensate the initial sensing value M₀ with the reference sensing value S, according to whether the initial sensing value M₀ determined by the sensor 110 is within the error range S±α of the reference sensing value S.

If the initial sensing value M₀ is not within the error range S±α of the reference sensing value S, the control unit 130 compensates the initial sensing value M₀ with the reference sensing value S, in step 440. By compensating the initial sensing value M₀ with the reference sensing value S, the control unit 130 may reduce a time taken for a sensing value determined by the sensor 110 to arrive at an actual value or a value within a certain range from the actual value.

In step 450, the sensor 110 determines a sensing value based on the compensated sensing value.

In operation 460, the control unit 130 determines whether the determined sensing value Mₙ is within the error range S±α of the reference sensing value S.

If the determined sensing value Mₙ is not within the error range S±α of the reference sensing value S, the control unit 130 determines whether a set time TM has elapsed.

If the set time TM has not elapsed, the process returns to step 450 so that determination of the sensing value may be repeated. The control unit 110 may repeat the determination of the sensing value at an interval of a specified time.

If the set TM has elapsed, the control unit 130 determines a most recently determined sensing value Mm as a final sensing value V, in step 480.

If the initial sensing value M₀ is within the error range S±α, of the reference sensing value S, in step 430, or if the determined sensing value Mₙ is within the error range S±α of the reference sensing value S, in step 460, after elapse of the set time TM, the control unit 130 determines the final sensing value V.

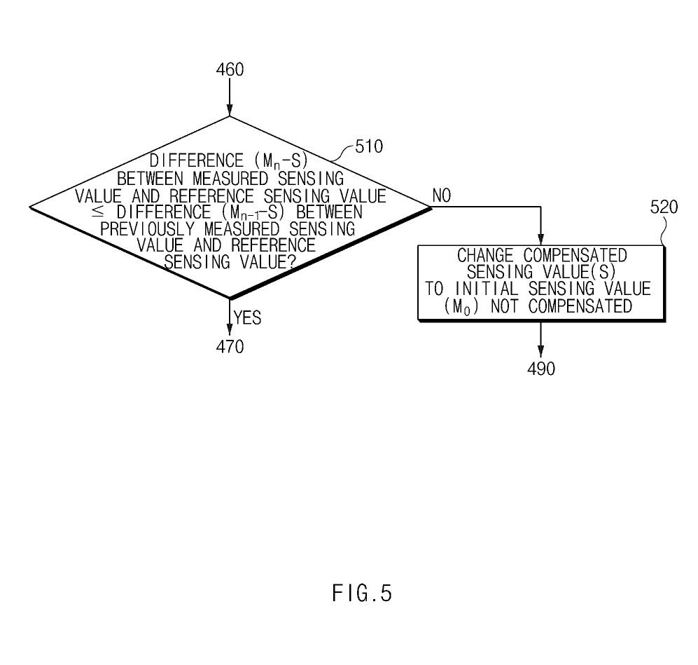

FIG. 5 is a flowchart illustrating a method for determining a sensing value, according to an embodiment of the present disclosure. The flowchart of FIG. 5 may be added to the position A of FIG. 4, according to an embodiment of the present invention.

In step 510, the control unit 130 determines whether a difference between the determined sensing value Mₙ and the reference sensing value S, i.e., Mₙ−S, is less than or equal to a difference between a previously determined sensing value Mₙ₋₁ and the reference sensing value S, i.e., Mₙ₋₁−S.

If the difference Mₙ−S is less than or equal to the difference Mₙ₋₁−S, the process proceeds to step 470 of FIG. 4.

If the difference Mₙ−S is greater than the difference Mₙ₋₁−S, the control unit 130 changes the compensated sensing value S into the initial sensing value M₀ not compensated. That is, if the difference Mₙ−S is greater than the difference Mₙ₋₁−S, the initial compensation of the reference sensing value S is not effective, and thus, the control unit 130 determines a sensing value in a state prior to the compensation operation.

After elapse of the set time TM, the control unit 130 determines the final sensing value V based on the initial sensing value M₀.

The above-described methods may be programmed to be executed by a computer, and may be implemented in a general digital computer which executes the program using a computer-readable medium. The computer-readable recording medium includes a magnetic storage medium (e.g., a ROM, a floppy disk, a hard disk, or the like) and an optical recording medium (e.g., a CD-ROM, a DVD, or the like).

Each of the above-described elements of the electronic device, according to various embodiments of the present invention, may be configured with one or more components, and the names of the elements may be changed according to the type of the electronic device. The electronic device, according to various embodiments of the present invention, may include at least one of the above-described elements, and some elements may be omitted or additional elements may be added. Furthermore, some of the elements of the electronic device, according to various embodiments of the present invention, may be combined with each other so as to form one entity, so that the functions of the elements may be performed in the same manner as before the combination.

The term “module” used herein may represent, for example, a unit including one or more combinations of hardware, software, and firmware. The term “module” may be interchangeably used with the terms “unit”, “logic”, “logical block”, “component” and “circuit”. The module may be a minimum unit of an integrated component or may be a part thereof. The module may be a minimum unit for performing one or more functions or a part thereof. The module may be implemented mechanically or electronically. For example, the module, according an embodiment to the present invention, may include at least one of an application-specific integrated circuit (ASIC) chip, a field-programmable gate array (FPGA), and a programmable-logic device for performing some operations, which are known or will be developed.

According to an embodiment of the present invention, at least a part of devices (e.g., modules or functions thereof) or methods (e.g., operations) may be implemented as instructions stored in a computer-readable storage medium in the form of a programming module. When the instructions are performed by at least one processor (e.g., the control unit 130), the at least one processor may perform functions corresponding to the instructions. The computer-readable storage medium may be, for example, the storage unit 140. At least a part of the programming module may be implemented (e.g., executed) by the control unit 130. At least a part of the programming module may include, for example, a module, program, routine, sets of instructions, or process for performing at least one function.

The computer-readable recording medium may include a magnetic medium such as a hard disk, a floppy disk and a magnetic tape, an optical medium such as a compact disk read only memory (CD-ROM) and a digital versatile disc (DVD), a magneto-optical medium such as a floptical disk, and a hardware device configured to store and execute program instructions (e.g., a programming module), such as a ROM, a RAM, a flash memory, or the like. The program instructions may include machine language codes generated by compilers and high-level language codes that can be executed by computers using interpreters. The above-described hardware may be configured to be operated as one or more software modules for performing operations of the present disclosure and vice versa.

The module or programming module, according to an embodiment the present invention, may include at least one of the above-described elements, or some elements may be omitted or other additional elements may be added. Operations performed by the module, the programming module or the other elements may be performed in a sequential, parallel, iterative, or heuristic way. Furthermore, some operations may be performed in another order or may be omitted, or other operations may be added.

According to an embodiment of the present invention, a reference sensing value received from an external device is used to determine a sensing value using a sensor installed in an electronic device, so that a measurement speed and accuracy of the sensor may be improved.

Furthermore, a time taken for a value determined by the sensor to arrive at an actual value may be reduced using the reference sensing value, even if the electronic device undergoes rapid environmental changes due to an increase in temperature of the electronic device, a temperature of a human body contacting the electronic device, or environmental conditions.

While the invention has been shown and described with reference to certain embodiments thereof, it will be understood by those skilled in the art that various changes in form and detail may be made therein without departing from the spirit and scope of the invention as defined by the appended claims.

What is claimed is:

1. A method for determining a sensing value, the method comprising the steps of:
receiving, at an electronic device, from an external device, a reference sensing value related to a sensor of the electronic device;
determining an initial sensing value by measuring a parameter with the sensor;
determining whether the initial sensing value is within an error range of the reference sensing value;
compensating the initial sensing value with the reference sensing value, if the initial sensing value is not within the error range;
determining, by the sensor, a sensing value based on the compensated sensing value; and
repeatedly determining the sensing value until the sensing value is within the error range or until a set time expires.

2. The method of claim 1, further comprising determining a most recently determined sensing value as a final sensing value if the set time expires.

3. The method of claim 1, further comprising determining a final sensing value based on the initial sensing value after the set time expires, if a difference between a currently determined sensing value and the reference sensing value is greater than a difference between a previously determined sensing value and the reference sensing value.

4. The method of claim 1, further comprising determining a final sensing value based on the initial sensing value after the set time expires, if the initial sensing value is within the error range.

5. The method of claim 1, further comprising determining a final sensing value based on a most recently determined sensing value after the set time expires, if the sensing value is within the error range.

6. The method of claim 1, wherein repeatedly determining the sensing value is performed at a specified time interval.

7. The method of claim 1, further comprising receiving a selection of a user regarding whether to use the reference sensing value.

8. The method of claim 1, further comprising:
receiving an input for the error range; and
setting the error range based on the input.

9. The method of claim 1, wherein:
the external device is a server, and
the reference sensing value is provided from the server based on at least one of a location, a date, a time, and a space in which the electronic device is positioned.

10. The method of claim 1, further comprising:
transmitting location information of the electronic device to the external device,
wherein the reference sensing value is determined based on the location information of the electronic device.

11. The method of claim 1, further comprising transmitting a final sensing value as the reference sensing value to another electronic device positioned within a specified distance from the electronic device.

12. The method of claim 1, wherein the sensor is at least one of a temperature sensor, a humidity sensor, and a temperature-humidity sensor.

13. An electronic device comprising:
a sensor configured to determine a sensing value by measuring a parameter;
a communication interface unit configured to receive a reference sensing value related to the sensor from an external device; and
a control unit configured to:
determine whether an initial sensing value determined by the sensor is within an error range of the reference sensing value,
compensate the initial sensing value with the reference sensing value, if the initial sensing value is not within the error range; and
control the sensor to repeatedly determine the sensing value based on the compensated sensing value until the sensing value is within the error range or until a set time expires.

14. The electronic device of claim 13, wherein the control unit is further configured to determine a most recently determined sensing value as a final sensing value if the set time expires.

15. The electronic device of claim 13, wherein, if a difference between a currently determined sensing value and the reference sensing value is greater than a difference between a previously determined sensing value and the reference sensing value, a final sensing value is determined based on the initial sensing value after the set time expires.

16. The electronic device of claim 13, wherein, if the initial sensing value is within the error range, a final sensing value is determined based on the initial sensing value after the set time expires.

17. The electronic device of claim 13, wherein, if the sensing value is within the error range, a final sensing value is determined based on a most recently determined sensing value after the set time expires.

18. The electronic device of claim 13, wherein the communication interface unit is further configured to transmit location information of the electronic device to the external device, and receive the reference sensing value determined based on the location information of the electronic device from the external device.

19. The electronic device of claim 13, wherein the communication interface unit is further configured to transmit a final sensing value as the reference sensing value to another electronic device positioned within a specified distance from the electronic device.

20. A sensor for determining a sensing value, the sensor comprising:
a measurement unit configured to measure a parameter;
a measurement processing unit configured to determine an initial sensing value of the measured parameter;
a calibration unit configured to compensate the initial sensing value with a reference sensing value, if the initial sensing value is not within an error range of the reference sensing value;
wherein the measurement processing unit repeatedly determines a sensing value based on the compensated sensing value until the sensing value is within the error range or until a set time expires.
